(19) United States
(12) Patent Application Publication
(10) Pub. No.: US 2011/0135723 A1
(43) Pub. Date: Jun. 9, 2011

(54) PHARMACEUTICAL COMPOSITIONS CONTAINING PREGABALIN

(75) Inventors: Rajesh KSHIRSAGAR, Bangalore (IN); Ganesh SHINDE, Bangalore (IN); Pravin KAMBLE, Bangalore (IN); Sachin MUNDADE, Bangalore (IN); S. M. MUDDA, Bangalore (IN)

(73) Assignee: MICRO LABS LIMITED, Bangalore (IN)

(21) Appl. No.: 12/818,060

(22) Filed: Jun. 17, 2010

Related U.S. Application Data
(63) Continuation of application No. PCT/IB2010/001397, filed on Jun. 10, 2010.

(30) Foreign Application Priority Data
Jun. 12, 2009 (IN) 1386/CHE/2009

(51) Int. Cl. A61K 31/195; A61K 9/20

(52) U.S. Cl. 424/465; 514/561

(57) ABSTRACT

The present invention provides pharmaceutical composition comprising pregabalin that is useful for once daily oral dosing. The present invention further relates to pharmaceutical composition comprising pregabalin that is useful for once daily oral dosing comprising pregabalin and on or more water insoluble components. The present invention preferably relates to a gastro-retentive tablet comprising pregabalin and one or more water insoluble component wherein water insoluble component preferably comprises of combination of ethylcellulose and hydrogenated castor oil.

### CROSS-REFERENCE TO A RELATED APPLICATION

This application is a continuation of PCT Patent Application Number PCT/IB2010/001397 filed Jun. 10, 2010 which claimed priority to Indian Patent Application Number 1386/CHE/2009 filed Jun. 12, 2009. The entire disclosures of these applications are expressly incorporated herein by reference.

### FIELD OF THE INVENTION

The present invention relates to a pharmaceutical composition, suitable for once daily dosing comprising pregabalin, or a pharmaceutically acceptable complex, salt, solvate or hydrate thereof.

### BACKGROUND OF THE INVENTION

Pregabalin, or (S)-(+)-3-aminomethyl-5-methyl-hexanoic acid, binds to the alpha-2-delta subunit of a calcium channel and is related to the endogenous inhibitory neurotransmitter delta aminobutyric acid (GABA), which is involved in the regulation of brain neuronal activity. Pregabalin exhibits anti-seizure activity, as discussed in U.S. Pat. No. 5,563,175 to R. B. Silverman et al., and is useful for treating, among other conditions, epilepsy, pain, physiological conditions associated with psychomotor stimulants, inflammation, gastrointestinal damage, alcoholism, insomnia, fibromyalgia, and various psychiatric disorders, including anxiety, depression, mania, and bipolar disorder. In the United States, pregabalin has been approved for the treatment of diabetic peripheral neuropathy, postherpetic neuralgia, and as an adjunctive treatment for partial onset seizures in adults.

Pregabalin is available as an immediate release (IR) formulation in capsules and is administered to patients' two- or three-times daily (BID or TID). Many patients receiving pregabalin or other drugs, which are, administered two or more times daily would likely benefit from once daily dosing. The convenience of once daily dosing generally improves patient compliance, especially for elderly patients and for patients taking multiple medications. Once per day dosing may also lessen or prevent potentially undesirable dose-related effects by reducing peak blood levels (C MAX) and may also increase drug efficacy by increasing minimum plasma concentrations (C_{MIN}).

Once daily dosing of pregabalin, however, presents numerous challenges. Conventional extended release (ER) compositions are problematic for dosing because pregabalin is not absorbed uniformly in the gastrointestinal (GI) tract. Clinical studies indicate that pregabalin is absorbed in the small intestine and the ascending colon in humans, but is poorly absorbed beyond the hepatic flexure. This suggests that the mean absorption window for pregabalin is, on average, about six hours or less and any drug release from a conventional ER dosage form beyond six hours would thus be wasted because the dosage form has traveled beyond the hepatic flexure.

United States patent application no. 2007/0269511 A1 discloses pregabalin formulations containing matrix forming agent and swelling agent wherein the matrix forming agent is polyvinyl acetate and polyvinylpyrrolidone, and the swelling agent is cross-linked polyvinylpyrrolidone.

Thus still there exists need in the art for robust, once daily dosage form for pregabalin.

### SUMMARY OF THE INVENTION

The present invention provides pharmaceutical composition comprising pregabalin that is useful for once daily oral dosing. When administered as a solid dosage form, such as tablet, the pharmaceutical composition is retained in the stomach for a longer period of time than an IR dosage form. While it is retained in the stomach, the pharmaceutical composition continuously releases pregabalin for more than 8 hours, preferably more that 12 hours, still preferably more than 20 hours. Extending the period of time during which pregabalin is released in the stomach effectively widens the absorption window associated with IR dosing, thereby permitting once daily dosing.

One aspect of the invention provides a pharmaceutical composition which is suitable for once daily dosing and includes an active pharmaceutical ingredient and excipients. The active pharmaceutical ingredient includes pregabalin, or a pharmaceutically acceptable complex, salt, solvate or hydrate thereof, and the excipients include one or more water insoluble component.

A further aspect of the invention provides a solid dosage form, such as a tablet, which is adapted for once daily oral dosing. The solid dosage form comprises the pharmaceutical composition described above.

A further aspect of the invention provides a floating tablet, which is adapted for once daily oral dosing. The floating tablet comprises the pharmaceutical composition described above.

An additional aspect of the invention provides a method of treating a condition or disorder in a subject that is responsive to pregabalin. The method includes orally administering to the subject once per day the pharmaceutical composition described above.

### BRIEF DESCRIPTION OF THE DRAWING

FIG. 1 shows dissolution profiles of the pharmaceutical compositions according to the present invention in 900 ml, 0.06 N HCl using USP type 2 apparatus at 50 rpm.

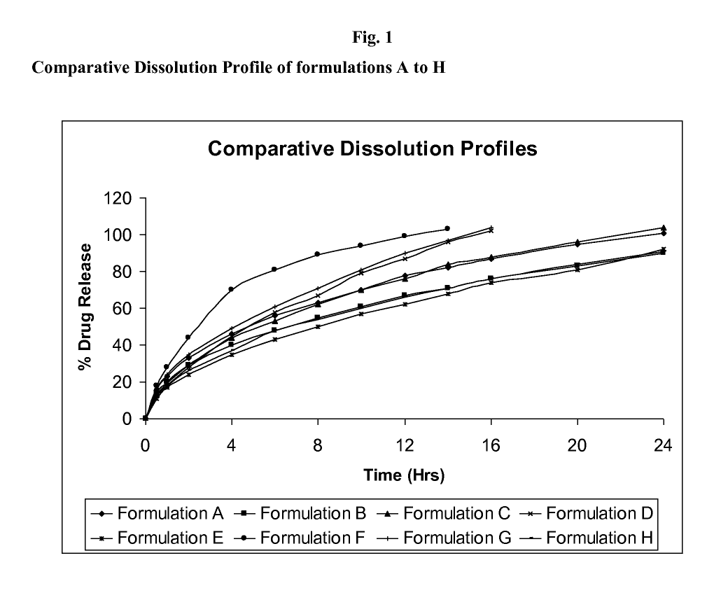

### DETAILED DESCRIPTION OF THE INVENTION

The present invention provides the pharmaceutical composition comprising pregabalin that is useful for once daily oral dosing.

In one embodiment, the present invention provides a pharmaceutical composition, which is suitable for once daily dosing comprising pregabalin, or a pharmaceutically acceptable complex, salt, solvate or hydrate thereof, and the excipients.

In yet another embodiment the present invention provides a pharmaceutical composition which is suitable for once daily dosing comprising pregabalin, or a pharmaceutically acceptable complex, salt, solvate or hydrate thereof, and one or more water insoluble component.

In yet another embodiment the present invention provides a pharmaceutical composition which is suitable for once daily dosing comprising pregabalin, or a pharmaceutically acceptable complex, salt, solvate or hydrate thereof, and one or more water insoluble component wherein pharmaceutical composition is retained in the stomach of subject following oral dosing for about 0 to 24 hours.

In yet another embodiment the present invention provides a pharmaceutical composition which is suitable for once daily dosing comprising pregabalin, or a pharmaceutically acceptable complex, salt, solvate or hydrate thereof, and one or more water insoluble component wherein the pregabalin is released over a period of time that is about 0 to 24 hours.

In yet another embodiment the present invention provides a pharmaceutical composition comprising pregabalin, or a pharmaceutically acceptable complex, salt, solvate or hydrate thereof, and one or more water insoluble component wherein pharmaceutical composition floats as soon as contacting biological and/or aqueous fluids and remains in floating state for more than 3 hours, still preferably for more than 6 hours, still preferably for more than 12 hours, still preferably for more than 18 hours, still preferably for more than 24 hours.

In yet another embodiment the present invention provides a pharmaceutical composition which is suitable for once daily dosing comprising pregabalin, or a pharmaceutically acceptable complex, salt, solvate or hydrate thereof, and one or more water insoluble component wherein the pharmaceutical composition can be bio-equivalent to an immediate release formulation comprising pregabalin, lactose monohydrate, maize starch, and talc.

In yet another embodiment the present invention provides a solid dosage form, such as a tablet, which is adapted for once daily oral dosing comprising pregabalin, or a pharmaceutically acceptable complex, salt, solvate or hydrate thereof, and one or more water insoluble component.

The water insoluble components includes but are not limited to water insoluble polymers selected from group ethyl cellulose, cellulose acetate, cellulose acetate phthalate, polymethyacrylates, calcium silicates and combinations thereof and the like; the waxes selected from hydrogenated castor oil, hydrogenated vegetable oil, bees wax, carnauba wax, microcrystalline wax, ozocarite, fatty acid esters such as glyceryl monostearate; glycerol monooleate, acetylated monoglycerides, tristearin, tripalmitin, cetyl esters wax, glyceryl palmitostearate, glyceryl behenate, zein, and combination thereof and the like. The most preferred water insoluble component is combination of ethylcellulose and hydrogenated castor oil.

In yet another embodiment the present invention provides a solid dosage form, such as a tablet, which is adapted for once daily oral dosing comprising pregabalin, or a pharmaceutically acceptable complex, salt, solvate or hydrate thereof, and water insoluble component wherein water insoluble component is combination of ethylcellulose and hydrogenated castor oil.

The present inventors have surprisingly found that the ratio of ethyl cellulose to hydrogenated castor oil is critical for preparing a gastro-retentive floating tablet suitable for once daily oral dosing comprising pregabalin wherein the floating and release of preagabalin in controlled manner is achieved by combination of ethyl cellulose and hydrogenated castor oil. Such gastro-retentive floating tablets floats as soon as contacting biological and/or aqueous fluids and remains in floating state for more than 3 hours, still preferably for more than 6 hours, still preferably for more than 12 hours, still preferably for more than 18 hours, still preferably for more than 24 hours wherein the weight ratio of ethyl cellulose to hydrogenated castor oil is from about 0.1:10 to about 10:0.1, preferably from about 0.5:10 to about 10:0.5, most preferably from about 1:5 to 5:1.

The present inventors have further found that also the ratio of ethyl cellulose to polyethylene glycol is critical for preparing a gastro-retentive floating tablet suitable for once daily oral dosing comprising pregabalin wherein the floating and release of preagabalin in controlled manner is achieved by combination of ethyl cellulose and polyethylene glycol. Such gastro-retentive floating tablets floats as soon as contacting biological and/or aqueous fluids and remains in floating state for more than 3 hours, still preferably for more than 6 hours, still preferably for more than 12 hours, still preferably for more than 18 hours, still preferably for more than 24 hours wherein the weight ratio of ethyl cellulose to polyethylene glycol is from about 0.1:10 to about 10:0.1, preferably from about 0.5:10 to about 10:0.5, most preferably from about 1:5 to 5:1.

In yet another embodiment the present invention provides a gastro-retentive floating tablet adapted for once daily oral dosing comprising pregabalin, or a pharmaceutically acceptable complex, salt, solvate or hydrate thereof, and one or more pharmaceutically acceptable excipients.

In yet another embodiment the present invention provides a gastro-retentive floating tablet which is adapted for once daily oral dosing comprising pregabalin, or a pharmaceutically acceptable complex, salt, solvate or hydrate thereof, and one or more pharmaceutically acceptable excipients wherein the tablet exhibits in vitro release of pregabalin not less than 30% after 4 hours.

In yet another embodiment the present invention provides a gastro-retentive floating tablet which is adapted for once daily oral dosing comprising pregabalin, or a pharmaceutically acceptable complex, salt, solvate or hydrate thereof, and one or more pharmaceutically acceptable excipients wherein the tablet exhibits in vitro release of pregabalin not less than 40% after 8 hours.

In yet another embodiment the present invention provides a gastro-retentive floating tablet which is adapted for once daily oral dosing comprising pregabalin, or a pharmaceutically acceptable complex, salt, solvate or hydrate thereof, and one or more pharmaceutically acceptable excipients wherein the tablet exhibits in vitro release of pregabalin not less than 50% after 12 hours.

In yet another embodiment the present invention provides a gastro-retentive floating tablet which is adapted for once daily oral dosing comprising pregabalin, or a pharmaceutically acceptable complex, salt, solvate or hydrate thereof, and one or more pharmaceutically acceptable excipients wherein the tablet exhibits in vitro release of pregabalin not less than 70% after 16 hours.

In yet another embodiment the present invention provides a gastro-retentive floating tablet which is adapted for once daily oral dosing comprising pregabalin, or a pharmaceutically acceptable complex, salt, solvate or hydrate thereof, and one or more pharmaceutically acceptable excipients wherein the tablet exhibits in vitro release of pregabalin not less than 85% after 24 hours.

In yet another embodiment the present invention provides a gastro-retentive floating tablet adapted for once daily oral dosing comprising pregabalin, or a pharmaceutically acceptable complex, salt, solvate or hydrate thereof, and combination of one or more water insoluble component and one or more water-soluble component.

The water soluble components include but are not limited to polyethylene glycol, hydroxypropyl methylcellulose, hydroxypropyl cellulose, methylcellulose, vinyl acetate copolymers, polysaccharides as alginates, xanthan gum, Chitosan, carrageenan, dextran and the like, polyalkylene oxides as polyethylene oxide and the likes, methaacrylic acid copolymers, maleic anhydride/methyl vinyl ether copolymers, carbomers and the like. The most preferred water-soluble component is polyethylene glycol.

In yet another embodiment the present invention provides a gastro-retentive floating tablet comprising pregabalin, or a pharmaceutically acceptable complex, salt, solvate or hydrate thereof, and combination of ethylcellulose and polyethylene glycol.

In yet another embodiment the present invention provides a gastro-retentive floating tablet adapted for once daily oral dosing comprising pregabalin, or a pharmaceutically acceptable complex, salt, solvate or hydrate thereof, and combination ethyl cellulose and polyethylene glycol wherein combination of ethyl cellulose and polyethylene glycol is present from about 1% w/w to about 80% w/w of the composition.

In yet another embodiment the present invention provides a gastro-retentive floating tablet adapted for once daily oral dosing comprising pregabalin, or a pharmaceutically acceptable complex, salt, solvate or hydrate thereof, and combination ethyl cellulose and polyethylene glycol wherein weight ratio of ethyl cellulose to polyethylene glycol is from about 0.1:10 to about 10:0.1, preferably from about 0.5:10 to about 10:0.5, most preferably from about 1:5 to 5:1.

In yet another embodiment the present invention provides a floating tablet comprising pregabalin, or a pharmaceutically acceptable complex, salt, solvate or hydrate thereof, and one or more water insoluble component.

In yet another embodiment present invention provides a gastro-retentive floating tablet adapted for once daily oral dosing comprising pregabalin, or a pharmaceutically acceptable complex, salt, solvate or hydrate thereof, and one or more water insoluble component wherein the tablet is capable of floating for 0 to 24 hours.

In yet another embodiment present invention provides a gastro-retentive floating tablet adapted for once daily oral dosing comprising pregabalin, or a pharmaceutically acceptable complex, salt, solvate or hydrate thereof, and one or more water insoluble component wherein the tablet is capable of floating for more that 20 hours.

In yet another embodiment the present invention provides a gastro-retentive floating tablet comprising pregabalin, or a pharmaceutically acceptable complex, salt, solvate or hydrate thereof, and water insoluble component wherein the water insoluble component comprises of combination of ethylcellulose and hydrogenated castor oil.

In yet another embodiment the present invention provides a gastro-retentive floating tablet which is adapted for once daily oral dosing comprising pregabalin, or a pharmaceutically acceptable complex, salt, solvate or hydrate thereof, and a combination of ethyl cellulose and hydrogenated castor oil; wherein weight ratio of ethyl cellulose to hydrogenated castor oil is from about 0.1:10 to about 10:0.1, preferably from about 0.5:10 to about 10:0.5, most preferably from about 1:5 to 5:1.

In yet another embodiment the present invention provides a gastro-retentive floating tablet which is adapted for once daily oral dosing comprising pregabalin, or a pharmaceutically acceptable complex, salt, solvate or hydrate thereof, and a combination of ethyl cellulose and hydrogenated castor oil wherein said combination of ethyl cellulose and hydrogenated castor oil is present from about 1% w/w to about 80% w/w of the composition.

In yet another embodiment the present invention provides a gastro-retentive floating bi-layer tablet comprising pregabalin, or a pharmaceutically acceptable complex, salt, solvate or hydrate thereof, wherein both the floating layer and the release controlling layer comprises of same one or more water insoluble component.

In yet another embodiment the present invention provides a gastro-retentive floating bi-layer tablet comprising pregabalin, or a pharmaceutically acceptable complex, salt, solvate or hydrate thereof, wherein the floating layer and the release controlling layer both prepared of same composition comprising a combination of ethylcellulose and hydrogenated castor oil.

In yet another embodiment the present invention provides a gastro-retentive floating bi-layer tablet comprising pregabalin, or a pharmaceutically acceptable complex, salt, solvate or hydrate thereof, wherein the floating layer comprises of gas entrapping swelling system and release controlling layer comprises of a combination one or more water insoluble component.

In yet another embodiment the present invention provides a gastro-retentive floating bi-layer tablet comprising pregabalin, or a pharmaceutically acceptable complex, salt, solvate or hydrate thereof, wherein the floating layer comprises of gas entrapping swelling system comprises of sodium bicarbonate, sodium alginate, microcrystalline cellulose and release controlling layer comprises of a combination ethylcellulose and hydrogenated castor oil.

In yet another embodiment the present invention provides a gastro-retentive floating tablet comprising pregabalin, or a pharmaceutically acceptable complex, salt, solvate or hydrate thereof, and one or more water insoluble component wherein the tablet is single layer tablet without additional floating layer and capable of both floating and providing controlled release of pregabalin for about 24 hours, preferably for about 20 hours, still preferably for about 12 hours, still more preferably for about 8 hours.

In yet another embodiment the present invention provides a gastro-retentive floating tablet comprising pregabalin, or a pharmaceutically acceptable complex, salt, solvate or hydrate thereof, and one or more pharmaceutically acceptable excipients wherein tablet has friability below 1% w/w.

In yet another embodiment the present invention provides a gastro-retentive floating tablet which is adapted for once daily oral dosing comprising pregabalin, or a pharmaceutically acceptable complex, salt, solvate or hydrate thereof, and a combination of ethylcellulose and hydrogenated castor oil; wherein the tablet is single layer tablet without additional floating layer and capable of both floating and providing controlled release of pregabalin for about 24 hours, preferably for about 20 hours, still preferably for about 12 hours, still more preferably for about 8 hours.

In yet another embodiment the present invention provides a process of preparing a pharmaceutical composition comprising pregabalin, or a pharmaceutically acceptable complex, salt, solvate or hydrate thereof, and one or more water insoluble components wherein process can be selected from direct compression, dry granulation, wet granulation and melt granulation.

In yet another embodiment the present invention provides a process of preparing gastro-retentive floating tablet comprising pregabalin, or a pharmaceutically acceptable complex, salt, solvate or hydrate thereof, one or more water insoluble component wherein the water insoluble component is combination of ethylcellulose hydrogenated castor oil prepared by melt granulation technique.

In yet another embodiment the present invention provides a method of treating a condition or disorder in a subject that is responsive to pregabalin the method comprising administering subject once daily pharmaceutical composition comprising pregabalin, or a pharmaceutically acceptable complex, salt, solvate or hydrate thereof, and one or more water insoluble component. The method includes orally administering to the subject once per day the pharmaceutical composition described above.

The dosage forms of the present invention typically contain 25 to 900 mg pregabalin as base. The dosage forms of the invention optionally may comprise pharmaceutically acceptable complexes, salts, polymorphs, hydrates, solvates, enantiomers or racemates of pregabalin.

“Pharmaceutical composition” refers to the combination of one or more drug substances and one or more excipients.

“Drug product,” “pharmaceutical dosage form,” “dosage form,” “final dosage form” and the like, refer to a pharmaceutical composition that is administered to a subject in need of treatment and generally may be in the form of tablets, capsules, sachets containing powder or granules and the like.

“Retained in the stomach or Gastro-retentive,” when used in connection with a pharmaceutical composition or dosage form, means that at least a portion of the dosage form remains in a subject's stomach following oral administration for about three or more hours, which is substantially longer than the average residence time of a corresponding IR dosage form. While it is retained in the stomach, the dosage form continuously releases the drug.

“Release,” “released,” and the like, when used in connection with a pharmaceutical composition or dosage form, refers to the portion of the drug substance that leaves the dosage form following contact with an aqueous environment. Unless otherwise indicated, the quantity of drug released from a dosage form is measured by dissolution testing in 900 ml, 0.06N HCl using apparatus USP type 2 at 50 rpm.

The compositions of the present invention can also include other materials such as binders, diluents, anti-adherents, glidants and lubricants.

Binders may be, for example, starch, sugars, gums, low molecular weight hydroxypropyl methylcellulose, povidone, hydroxypropyl cellulose, hydroxyethyl cellulose or the like.

Diluents may be, for example, any pharmaceutically acceptable, non-toxic diluent. Particular examples include lactose, dextrose, sucrose, maltose, microcrystalline cellulose, starch, calcium hydrogen phosphate, mannitol and the like.

Anti-adherents may be, for example, silicon-containing compounds such as silicon dioxide, magnesium trisilicate, talc and the like.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and the like.

Lubricants may be, for example, talc, magnesium stearate, calcium stearate, stearic acid, sodium stearyl fumarate, sodium benzoate or the like. Antiadherents and Glidants may be, for example, colloidal silicon dioxide, talc or the like.

Solid oral dosage forms of the present invention may be prepared by any conventional techniques for example dry granulation, direct compression, wet granulation, melt granulation and extrusion-spheronization.

The following examples are intended to be illustrative and non-limiting:

### Example 1

### Bi-Layer Floating Tablet (Non-Gas Generating or Non-Swelling Floating Layer)

TABLE NO. 1

| Formulation Name | | | |
|---|---|---|---|
| A | B | C | D |

| | |
|---|---|
| Ingredients | Qty/Tab (mg) |

A) Release Controlling layer Composition
Intra Granular

| | | | | |
|---|---|---|---|---|
| Pregabalin | 600.0 | 600.0 | 600.0 | 600.0 |
| Ethyl Cellulose | 50.0 | 46.0 | 50.0 | 50.0 |
| Hydrogenated Castor Oil | 100.0 | 100.0 | 50.0 | 75.0 |

Extra Granular

| | | | | |
|---|---|---|---|---|
| Ethyl Cellulose | 46.0 | — | 46.0 | 46.0 |
| Magnesium Stearate | 4.0 | 4.0 | 4.0 | 4.0 |
| Total Weight of CR Layer | 800.0 | 750.0 | 750.0 | 775.0 |

B) Floating Layer Composition

| | | | | |
|---|---|---|---|---|
| Ethyl Cellulose | 305.0 | 305.0 | 152.5 | 152.5 |
| Hydrogenated Castor Oil | 190.0 | 190.0 | 95.0 | 95.0 |
| Magnesium Stearate | 5.0 | 5.0 | 2.5 | 2.5 |
| Total Weight of Floating Layer | 500.0 | 500.0 | 250.0 | 250.0 |
| Total Weight of Tablets | 1300.0 | 1250.0 | 1000.0 | 1025.0 |

### Manufacturing Process:

The bi-layer tablets were prepared using formula as described in Table No. 1. The manufacturing process used is as follows:

### A] Preparation of Controlled Release Granules

The Hydrogenated castor oil was melted at temperature about 90° C. Further pregabalin or mixture of pregabalin and part of Ethyl cellulose was sieved and mixed with melted hydrogenated castor oil. Above mixture is cooled with stirring and sieved to obtain granules.

Obtained granules were mixed with ethylcellulose (if applicable). This mixture was lubricated using magnesium stearate.

### B) Preparation of Floating Layer

The ethylcellulose and Hydrogenated castor oil were sieved and mixed thoroughly. This mixture was lubricated using magnesium stearate.

### C) Preparation of Bi-Layer Tablets

The bi-layer tablets were prepared using 21.0×11.0 mm oval shaped, standard concave punches suitable for tablet compression machine.

The tablets obtained above were tested for dissolution in 900 ml 0.06 NHCL using USP II (Paddle) apparatus at 50 rpm at 37° C.±0.5. The results obtained are given in Table No. 2

TABLE NO. 2

| | | | |
|---|---|---|---|
| A | B | C | D |

| | |
|---|---|
| Time (hrs) | Cumulative % Drug release |

| | | | | |
|---|---|---|---|---|
| 0.5 | 16 | 14 | 13 | 12 |
| 1 | 23 | 20 | 19 | 18 |
| 2 | 33 | 29 | 29 | 28 |
| 4 | 46 | 40 | 44 | 45 |
| 6 | 56 | 48 | 53 | 58 |
| 8 | 63 | 55 | 62 | 67 |
| 10 | 70 | 61 | 70 | 79 |
| 12 | 78 | 67 | 76 | 87 |
| 14 | 82 | 71 | 84 | 96 |
| 16 | 87 | 76 | 88 | 102 |
| 20 | 95 | 83 | 96 | — |
| 24 | 101 | 90 | 104 | — |

### Example 2

### Bi-Layer Floating Tablet (Gas Generating or Swelling Floating Layer)

TABLE NO. 3

| | |
|---|---|
| | Formulation Name |
| | E |
| Ingredients | Qty/Tab (mg) |

A) Release controlling layer Composition
Intra Granular

| | |
|---|---|
| Pregabalin | 600.0 |
| Ethyl Cellulose | 50.0 |
| Hydrogenated Castor Oil | 100.0 |

Extra Granular

| | |
|---|---|
| Ethyl Cellulose | 46.0 |
| Magnesium Stearate | 4.0 |
| Total Weight of CR Layer | 800.0 |

B) Floating Layer Composition

| | |
|---|---|
| Sodium Bicarbonate | 107.0 |
| Sodium Alginate | 212.0 |
| Microcrystalline Cellulose | 181.0 |
| Total Weight of Floating Layer | 500.0 |
| Total Weight of Tablets | 1300.0 |

### Manufacturing Process:

The bi-layer tablets were prepared using formula as described in Table No. 3 the manufacturing process used was as follows:

### A] Preparation of Release Controlled Granules

The Hydrogenated castor oil was melted at temperature about 90° C. Further pregabalin or mixture of pregabalin and part of Ethyl cellulose was sieved and mixed with melted hydrogenated castor oil. Above mixture is cooled with stirring and sieved to obtain granules.

Obtained granules were mixed with ethylcellulose. This mixture was lubricated using magnesium stearate.

### B) Preparation of Floating Layer

The Sodium Bicarbonate, Sodium Alginate and Microcrystalline Cellulose were sieved and mixed thoroughly.

C) Preparation of Bi-Layer Tablets

The bi-layer tablets were prepared using 21.0×11.0 mm oval shaped, standard concave punches suitable for tablet compression machine.

The tablets obtained above were tested for dissolution in 900 ml 0.06 NHCL using USP II (Paddle) apparatus at 50 rpm at 37° C.±0.5. The results obtained are given in Table No. 4

TABLE NO. 4

| | E |
|---|---|
| Time (hrs) | Cumulative % Drug release |
| 0.5 | 11 |
| 1 | 17 |
| 2 | 24 |
| 4 | 35 |
| 6 | 43 |
| 8 | 50 |
| 10 | 57 |
| 12 | 62 |
| 14 | 68 |
| 16 | 74 |
| 20 | 81 |
| 24 | 92 |

### Example 3

### Single Floating tablet

TABLE NO. 5

| Formulation Name | | |
|---|---|---|
| F | G | H |

| | |
|---|---|
| Ingredients | Qty/Tab (mg) |

Intra Granular

| | | | |
|---|---|---|---|
| Pregabalin | 600.0 | 600.0 | 600.0 |
| Ethyl Cellulose | 50.0 | 50.0 | 50.0 |
| Hydrogenated Castor Oil | 50.0 | 100.0 | 100.0 |

Extra Granular

| | | | |
|---|---|---|---|
| Ethyl Cellulose | 46.0 | 46.0 | 198.5 |
| Hydrogenated Castor Oil | — | — | 95.0 |
| Magnesium Stearate | 4.0 | 4.0 | 6.5 |
| Total Weight of Tablets | 750.0 | 800.0 | 1050.0 |

### Manufacturing Process:

The floating tablets were prepared using formula as described in table No. 5 the manufacturing process used was as follows:

The Hydrogenated castor oil was melted at temperature about 90° C. Further pregabalin or mixture of pregabalin and part of Ethyl cellulose was sieved and mixed with melted hydrogenated castor oil. Above mixture is cooled with stirring and sieved to obtain granules. Obtained granules were mixed with ethylcellulose and hydrogenated castor oil (if applicable). This mixture was lubricated using magnesium stearate. The tablets were prepared using suitable tablet compression machine.

The tablets obtained above were tested for dissolution in 900 ml 0.06 NHCL using USP II (Paddle) apparatus at 50 rpm at 37° C.±0.5. The results obtained are given in Table No. 6

TABLE NO. 6

| | F | G | H |
|---|---|---|---|
| Time (hrs) | Cumulative % Drug release | | |
| 0.5 | 18 | 16 | 12 |
| 1 | 28 | 24 | 18 |
| 2 | 44 | 35 | 26 |
| 4 | 70 | 49 | 37 |
| 6 | 81 | 61 | 48 |
| 8 | 89 | 71 | 54 |
| 10 | 94 | 81 | 60 |
| 12 | 99 | 90 | 66 |
| 14 | 103 | 97 | 71 |
| 16 | — | 104 | 76 |
| 20 | — | — | 84 |
| 24 | — | — | 91 |

### Example 4

### Single Floating tablet

TABLE NO. 7

| | |
|---|---|
| | Formulation Name |
| | I |
| Ingredients | Qty/Tab (mg) |

Intra Granular

| | |
|---|---|
| Pregabalin | 600.0 |
| Ethyl Cellulose | 50.0 |
| Polyethylene Glycol 6000 | 100.0 |
| Ethanol | q.s. |

Extra Granular

| | |
|---|---|
| Ethyl Cellulose | 145.0 |
| Polyethylene Glycol 6000 | 100.0 |
| Magnesium Stearate | 5.0 |
| Total Weight of Tablet | 1000.0 |

### Manufacturing Process:

The floating tablets were prepared using formula as described in table No. 7 the manufacturing process used was as follows:

Preagabalin and intregranular quantity of ethyl cellulose, PEG 6000 were sieved and mixed. Sufficient quantity of ethanol was added above mixture and granules were prepared. Obtained granules were dried and sieved. Remaining quantity of ethyl cellulose, PEG 6000 was added to above granules mixed. This mixture was lubricated using magnesium Stearate. The tablets were prepared using suitable tablet compression machine.

The tablets obtained above were tested for dissolution in 900 ml 0.06 NHCL using USP II (Paddle) apparatus at 50 rpm at 37° C.±0.5. The results obtained are given in Table No. 8

TABLE NO. 8

| | I |
|---|---|
| Time (hrs) | Cumulative % Drug release |
| 0.5 | 15 |
| 1 | 23 |
| 2 | 34 |
| 4 | 47 |
| 6 | 58 |
| 8 | 66 |
| 10 | 74 |
| 12 | 81 |
| 14 | 85 |
| 16 | 91 |
| 20 | 99 |
| 24 | 105 |

We claim:

1. A gastro-retentive floating tablet adapted for once daily oral dosing comprising pregabalin, or a pharmaceutically acceptable complex, salt, solvate or hydrate thereof, and one or more pharmaceutically acceptable excipients.

2. A gastro-retentive floating tablet adapted for once daily oral dosing comprising pregabalin, or a pharmaceutically acceptable complex, salt, solvate or hydrate thereof, and one or more water insoluble components.

3. A gastro-retentive floating tablet according to claim 2 wherein water insoluble component is combination of ethyl cellulose and hydrogenated castor oil.

4. A gastro-retentive floating tablet according to claim 3 wherein weight ratio of ethyl cellulose to hydrogenated castor oil is from about 0.1:10 to about 10:0.1, preferably from about 0.5:10 to about 10:0.5, most preferably from about 1:5 to 5:1.

5. A gastro-retentive floating tablet according to claim 3 wherein combination of ethyl cellulose and hydrogenated castor oil is present from about 1% w/w to about 80% w/w of the composition.

6. A gastro-retentive floating tablet adapted for once daily oral dosing comprising pregabalin, or a pharmaceutically acceptable complex, salt, solvate or hydrate thereof, and combination of one or more water-insoluble component and one or more water-soluble component.

7. A gastro-retentive floating tablet according to claim 6 wherein tablet comprises of combination of ethyl cellulose and polyethylene glycol.

8. A gastro-retentive floating tablet according to claim 7 wherein weight ratio of ethyl cellulose to polyethylene glycol is from about 0.1:10 to about 10:0.1, preferably from about 0.5:10 to about 10:0.5, most preferably from about 1:5 to 5:1.

9. A gastro-retentive floating tablet according to claim 7 wherein combination of ethyl cellulose and polyethylene glycol is present from about 1% w/w to about 80% w/w of the composition.

10. A gastro-retentive floating bi-layer tablet adapted for once daily oral dosing comprising
a) floating layer
b) release controlling layer containing pregabalin, or a pharmaceutically acceptable complex, salt, solvate or hydrate thereof; and
c) pharmaceutically acceptable excipients.

11. A gastro-retentive floating tablet according to claim 10 wherein release-controlling layer comprises of combination of ethylcellulose and hydrogenated castor oil.

12. A gastro-retentive floating tablet according to claim 10 wherein release controlling layer comprises of combination of ethylcellulose and polyethylene glycol.

13. A gastro-retentive floating tablet according to claim 10 wherein the floating layer comprises of combination of ethylcellulose and hydrogenated castor oil.

14. A gastro-retentive floating tablet according to claim 10 wherein the floating layer comprises of gas entrapping swelling system.

15. A gastro-retentive floating tablet according to claim 11 wherein weight ratio of ethyl cellulose to hydrogenated castor oil is from about 0.1:10 to about 10:0.1, preferably from about 0.5:10 to about 10:0.5, most preferably from about 1:5 to 5:1.

16. A gastro-retentive floating tablet according to claim 11 wherein combination of ethyl cellulose and hydrogenated castor oil is present from about 1% w/w to about 80% w/w of the composition.

17. A gastro-retentive floating tablet according to claim 12 wherein weight ratio of ethyl cellulose to polyethylene glycol is from about 0.1:10 to about 10:0.1, preferably from about 0.5:10 to about 10:0.5, most preferably from about 1:5 to 5:1.

18. A gastro-retentive floating tablet according to claim 12 wherein combination of ethyl cellulose and polyethylene glycol is present from about 1% w/w to about 80% w/w of the composition.

19. A gastro-retentive floating tablet according to claim 1 wherein the tablet exhibits in vitro release of pregabalin not less than 30% after 4 hours.

20. A gastro-retentive floating tablet according to claim 1 wherein the tablet exhibits in vitro release of pregabalin not less than 40% after 8 hours.

21. A gastro-retentive floating tablet according to claim 1 wherein the tablet exhibits in vitro release of pregabalin not less than 50% after 12 hours.

22. A gastro-retentive floating tablet according to claim 1 wherein the tablet exhibits in vitro release of pregabalin not less than 70% after 16 hours.

23. A gastro-retentive floating tablet according to claim 1 wherein the tablet exhibits in vitro release of pregabalin not less than 85% after 24 hours.

24. A gastro-retentive floating tablet adapted for once daily oral dosing comprising pregabalin, or a pharmaceutically acceptable complex, salt, solvate or hydrate thereof, and one or more pharmaceutically acceptable excipients wherein tablet floats as soon as contacting biological and/or aqueous fluids and remains in floating state more than 3 hours.

25. A gastro-retentive floating tablet according to claim 24 wherein the tablet floats as soon as contacting biological and/or aqueous fluids and remains in floating state more than 6 hours.

26. A gastro-retentive floating tablet according to claim 24 wherein tablet floats as soon as contacting biological and/or aqueous fluids and remains in floating state more than 12 hours.

27. A gastro-retentive floating tablet according to claim 24 wherein tablet floats as soon as contacting biological and/or aqueous fluids and remains in floating state more than 24 hours.

28. A gastro-retentive floating tablet according to claim 1 wherein tablet has friability below 1% w/w.

29. A gastro-retentive floating tablet adapted for once daily oral dosing comprising pregabalin, or a pharmaceutically acceptable complex, salt, solvate or hydrate thereof, and one or more pharmaceutically acceptable excipients wherein the tablet is prepared by direct compression or dry granulation or wet granulation or melt granulation.

30. A method of treating a condition or disorder in a subject that is responsive to pregabalin the method comprising administering subject a gastro-retentive floating tablet adapted for once daily oral dosing comprising pregabalin, or a pharmaceutically acceptable complex, salt, solvate or hydrate thereof, and one or more pharmaceutically acceptable excipients.
